(19) United States
(12) Patent Application Publication
(10) Pub. No.: US 2008/0249420 A1
(43) Pub. Date: Oct. 9, 2008

(54) Vascular Catheter Device and Related Methods of Using the Same

(76) Inventor: Arthur W. Crossman, Daytona Beach, FL (US)

Correspondence Address: Robert J Decker, 2679 Teakwood Drive, Charlottesville, VA, 22911, US

(21) Appl. No.: 10/577,118

(22) PCT Filed: Oct. 14, 2005
(86) PCT No.: PCT/US05/37031

§ 371 Date: Apr. 26, 2006

Related U.S. Application Data
(60) Provisional application No. 60/618,695, filed on Oct. 14, 2004.

(51) Int. Cl. A61M 25/10; A61B 5/02; A61M 25/01

(52) U.S. Cl. 600/481; 604/96.01; 604/523

(57) ABSTRACT

A catheter device fur diagnostic vascular treatment and/or therapeutic vascular treatment of a subject's vasculature having a blunt, atraumatic (non-traumatic) distal tip and orifice edge to the catheter. This will eliminate or mitigate catastrophic complications caused by contemporary catheters such as vascular dissection, thrombosis, distal embolization and vessel occlusion. The catheter tip can be shaped in various ways with solid material (compressible or non-compressible) or balloon inflation devices to create a blunt, atraumatic (non-traumatic) tip and orifice, and may include a set-back region as well.

### CROSS-REFERENCE TO RELATED APPLICATIONS

The present invention claims priority under 35 U.S.C. Section 119(e) of the earlier filing date of U.S. Provisional Application Ser. No. 60/618,695, filed Oct. 14, 2004, entitled “Vascular Catheter Device and Related Method of Making and Using the Same,” which is hereby incorporated by reference herein in its entirety.

### BACKGROUND OF THE INVENTION

Diagnostic vascular catheterization is a classification of invasive procedures in which a catheter and related are passed into a peripheral vein or artery, through the blood vessels, and into the heart or other vasculature. These procedures permit the study of the heart chambers and the arteries supplying the heart or other vasculatures of the body to diagnose illness or disease. Some examples of diagnostic vascular catheterization are, but not limited thereto, are coronary and peripheral vascular (e.g., renal artery, iliofemoral, aortic, cerebrovascular) angiography (or coronary arteriogaphy and angiography).

Therapeutic vascular catheterization (i.e., interventional catheterization) is a classification of invasive procedures in which a catheter and related are passed into a peripheral vein or artery, through the blood vessels, and into the heart or other vasculature. These procedures are intended primarily for the treatment of cardiac illness and disease as well as other vasculature illnesses and diseases. Often the goals of therapeutic vascular catheterization (interventional catheterization) have some similarities to diagnostic catheterization, except the goal is placement of the catheter to treat an underlying condition. Some examples of therapeutic cardiac catheterization are, but not limited thereto, percutaneous transluminal angioplasty (PTA) (alternatively, percutaneous transluminal coronary angioplasty (PTCA)), percutaneous coronary intervention (PCI), and percutaneous transluminal interventions (PTI). Interventional catherization to include, for example, all transluminal mechanisms of vascular lumen enlargement.

Some drawbacks that are associated with the various diagnostic and therapeutic vasculature catheterizations are, but not limited thereto, the unnecessary complications that can occur and restricted operations related to advancing or moving the catheter shaft and catheter tip. For example, the edge of the catheter tip puts pressure on the shoulder of the plaque thus rupturing or injuring the plaque shoulder. As a result this can, for example, release contents of plaque and lead to thrombosis or allow contrast to track between layers of artery (or vein) causing a dissection. Further, if a wire is passed through a catheter then dissection can also occur if the catheter is pushed further forward whereby a layer of the vasculature can be further separated leading to thrombosis and dissection. Alternatively, while traversing any vascular structure atherosclerotic debris can be dislodged thus leading to embolization of debris and distal vessel occlusion. Accordingly, the amount of force and leverage applied to a catheter is compromised because of the aforementioned and other risks and complications.

There is therefore a need in the art for a more effective and safer method of practicing diagnostic and therapeutic vasculature catheterizations.

### BRIEF SUMMARY OF INVENTION

Conventional diagnostic and therapeutic arterial vascular catheters have leading edges that are circular with a non-blunt edge similar in shape to a drinking straw, for example. This may lead to vascular trauma due the relatively sharp edge of the catheter disrupting the layers of the vessel Or plaque shoulder secondary to pressure exerted and or inherent angulation of the edge interface with the vessel wall.

The various embodiments of the present invention are to provide a blunt, atraumatic (non-traumatic) distal tip and orifice edge to the catheter. This will eliminate or mitigate catastrophic complications caused by contemporary catheters such as vascular dissection, thrombosis, distal embolization and vessel occlusion. The various embodiments of the present invention catheter tip can be shaped in various ways with solid material (compressible or non-compressible) or balloon inflation devices to create a blunt, atraumatic (non-traumatic) tip and orifice. The shape can take on numerous forms including, but not limited to olive, bulbous, rounded, spherical, hemispherical, conical, oval, tapered, beveled, chamfered, graduated, ring, tubular, cylinder and/or multi-faceted like a cut diamond (as well as dodecaliedron, semi-dodecahedron, icosahedron, or semi- icosahedron, etc.). The present invention device and method would enable atraumatic intubation of all vascular structures. Some advantages associated with some of the embodiments include, but not limited thereto, elimination or mitigation of risks of the aforementioned complications as compared to conventional catheters and provide more aggressive intubation of vascular structures for improved leverage for delivery of therapeutic interventional hardware (i.e. balloons, stents, atherectomy devices, lasers, thrombectomy devices, etc.) in situations where conventional catheters may traumatize the vessel or fail to deliver the therapeutic hardware; secondary to concern for risk of vascular trauma and the aforementioned complications.

An aspect of an embodiment of the present invention provides a catheter device for diagnostic vascular treatment and/or therapeutic vascular treatment of a subject's vasculature. The catheter device comprising: a catheter shaft having a proximal portion and a distal portion; and a distal tip disposed on the distal portion, the distal tip having a blunt shape adapted to avoid or mitigate trauma with an ostium of the vasculature. The distal tip may further comprises a set-back extension located on the distal end of the distal tip.

An aspect of an embodiment of the present invention provides a method of performing diagnostic vascular treatment and/or therapeutic vascular treatment on a subject's vasculature using a catheter device. The catheter device may comprise: a catheter shaft having a proximal portion and a distal portion; and a distal tip disposed on the distal portion, the distal tip having a blunt shape adapted to avoid or mitigate trauma with an ostium of the vasculature. The distal tip may further comprises a set-back extension located on the distal end of the distal tip.

These and other aspects of the disclosed technology and systems, along with their advantages and features, will be made more apparent from the description, drawings and claims that follow.

### BRIEF SUMMARY OF THE DRAWINGS

The foregoing and other objects, features and advantages of the present invention, as well as the invention itself, will be more fully understood from the following description of preferred embodiments, when read together with the accompanying drawings, in which:

FIG. 1(A) illustrates a schematic elevation view of an embodiment of the present invention catheter device including a distal tip.

FIGS. 1(B)-1(D) illustrate schematic partial views of the catheter device of FIG. 1(A) with alternative embodiments of the distal tip.

FIG. 2 illustrates a schematic elevation and partial view of an embodiment of the present invention catheter device including a distal tip having a non-inflatable ring or rim (as well as other applicable contours mentioned herein).

FIG. 3 illustrates a schematic elevation and partial view of an embodiment of the present invention catheter device including a distal tip having an inflatable ring or rim (as well as other applicable contours mentioned herein).

FIGS. 4(A)-(B) illustrate schematic elevation and partial views of an embodiment of the present invention catheter device including a distal tip having a balloon in a non-inflated state and an inflated state, respectfully.

FIGS. 4(C)-(D) illustrate schematic elevation and partial views of an embodiment of the present invention catheter device including a distal tip having a set-back feature/segment extending from the very end of the distal tip, which can be applied to any of the present invention devices discussed herein.

FIGS. 5(A)-(B) illustrate schematic elevation and partial views of an embodiment of the present invention catheter device including a distal tip having a catheter tip in a non-compressed state and compressed state, respectfully.

FIG. 6(A) is a schematic elevation view of a sheath that has been inserted into a vasculature structure such as an artery, vein, or the like.

FIG. 6(B) is the sheath as shown in FIG. 6(A) with the catheter device extending there through.

FIGS. 7(A)-(B) illustrate schematic elevation and partial views of an embodiment of the present invention multi-balloon (or multi-compartment of a balloon) tipped diagnostic and therapeutic vascular catheter in the non-inflated and inflated state, respectfully.

FIGS. 8(A)-(B) illustrate schematic elevation and partial views of an embodiment of the present invention of a single or multi-balloon (or multi-compartment of a balloon or multi-balloon) orifice ring/rim diagnostic and therapeutic vascular catheter in the non-inflated and inflated state, respectfully.

FIG. 8(C) illustrates a schematic end view of FIG. 8(B). FIG. 8(D)) illustrates a schematic partial side view of FIG. 8(B).

### DETAILED DESCRIPTION OF THE INVENTION

Some of the embodiments of the present invention provide a diagnostic vascular catheter for imaging or a therapeutic vascular catheter for vascular lumen enlargement that is atraumatic by providing, among other things, the tip of the catheter blunt in shape. It should be appreciated that the embodiments of the present invention distal tip can be utilized with the existing arterial vascular imaging and therapeutic catheters in terms of their proximal portion of the catheter and shaft shape of the catheter with regards to design, and materials. It should be appreciated that some of the embodiments of the present invention distal tip and balloons can utilize materials available in the field.

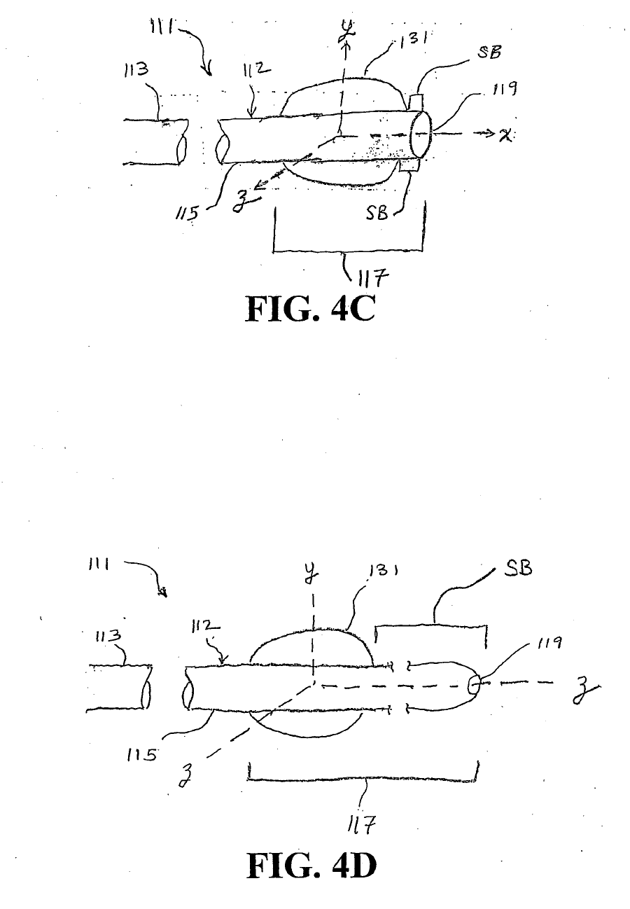

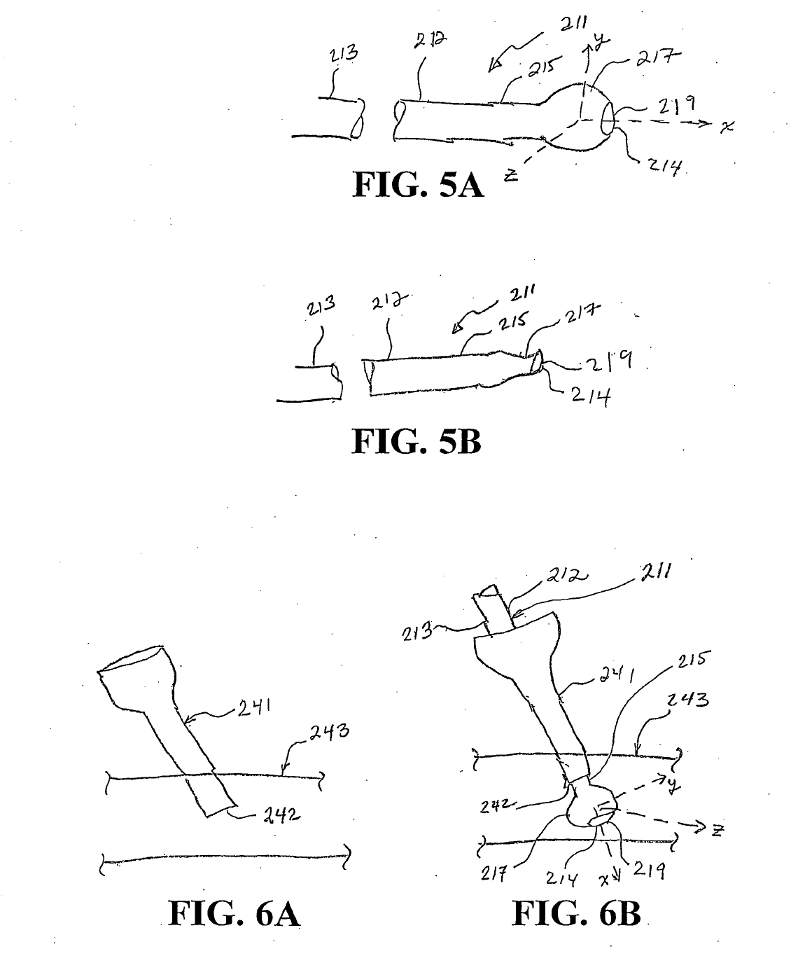

In an embodiment of the present invention catheter, the distal tip and tip orifice includes, but not limited thereto, the following: 1) solid non-compressible blunt tip and orifice/orifice edge (perimeter, partial perimeter or the like). The material of the non-compressible tip can be composed of some material currently available for catheter tips and orifice leading edges, as well as any other suitable non-compressible material that is suitable for a catheter. The leading orifice edge (e.g. perimeter, partial perimeter or the like) may be rounded toward the catheter lumen creating a smooth, non-edged orifice interface with the leading contacted portion(s) of the endovascular luminal wall tissue. The outer portion of the orifice may also be smooth and blunt in shape and gently flared toward the more proximal portion of the distal tip. This will create a smooth, blunt and atraumatic interface with the non-leading more proximal portion(s) of the contacted endovascular wall tissue. In short, the catheter orifice and distal tip will be shaped much like an olive, blunt tipped cone, sphere, hemisphere, etc. The x, y and z planes as well as the angle of curvature of the proximal and distal flared surfaces of the distal tip can be manipulated along the entire geometric spectrum of potential shapes to create a relatively spherical, olive shaped or conical shaped structure. The angle of curvature of the proximal and distal flared surfaces can also be manipulated along the entire geometric spectrum of curvature to create a relatively more blunt, conical, faceted or angulated structure. The luminal geometry of the catheter tip may remain unchanged in order to accommodate existing contemporary vascular luminal enlarging devices and/or contrast, drugs, fluid, etc. For example, 4 french would remain 4 french, 5 french would remain 5 french, etc. Because a flared tip may be a larger French size than the remainder of the catheter shaft, a larger vascular sheath would be required. Up sizing the vascular sheath may be avoided by the following adjustments to the catheter tip and orifice while at the same time maintaining the same geometry as described above. First, the flared distal tip is compressible so for instance if the flared tip is 7 french it can be compressed through a 6 french sheath and reform its atraumatic blunt geometry after traversing the vascular sheath (See for example, FIGS. 5-6). Second, the distal tip and orifice edge (perimeter or near perimeter or the like) are shaped by an inflatable balloon(s) which may be commence in a deflated state and can be inflated after traversing the vascular sheath and deflated upon removal of the catheter through the vascular sheath(See for example, FIGS. 4, and 6-8). The balloon is inflated by a separate lumen connected to an inert gas, radiographic contrast, fluid or air delivery system at the operator end of the catheter, for example. Separate lumens, balloons, compartments and inflation devices would be required for separate manipulation of x, y and z planes with a larger covering balloon or balloon like material or membrane covering the three x, y and z plane balloons. This would enable more detailed and/or variable shape changes as further elaborated later in the text. Alternatively a “covering” balloon (e.g., outer membrane) could be optional and/or alternatively a balloon could have a pre-formed shape with only size of the balloon being able to be controlled by the operator. Size of the balloons could be a function of balloon material compliance and inflation pressure as in contemporary PTCA balloon material for example.

It should be appreciated that the distal tips, set-backs, lumens, balloons, compartments may provide volume contoured according to desired size, shape and position for a particular ostia or vascular (e.g., location or anatomy) for a given procedure or treatment.

It should be appreciated that any catheter device/system discussed herein may be single lumen or multi-lumen.

As previously mentioned the geometry of the balloon catheter tip and orifice can take on all shapes along the entire continual geometric spectrum of manipulation of x, y and z planes of the catheter distal tip and orifice to create a relatively conical, olive, ellipsoid, hemispherical, tubular, ring, cylindrical, multifaceted or spherical shape with changing of the long and short axes as well as the angle of curvature of the proximal and distal flared surfaces. Size of the balloon tip could also be manipulated by varying the compliance of the balloon material and inflation pressure. Alternatively, the orifice edge (perimeter or near perimeter or the like) itself can exclusively be shaped by solid means (compressible or non-compressible) or balloon inflational means as an atraumatic blunt ring. The ring would be somewhat analogous to an innertube covering the metal edge of a wheel, for example. A difference being that in this case the ring would cover the leading edge of the distal tip orifice. Whether solid or inflatable, compressible or non-compressible the geometry of the ring could be manipulated changing the axes in x, y and z planes (as detailed above) of the ring inner diameters and lengths respectively to create the entire geometric spectrum of shapes capable with these manipulations. Again this would require separate lumina, with separate inflation devices corresponding to separate balloons in x, y and z planes for the desired effect of shape manipulation. These separate balloons could be covered by a covering balloon material (e.g., outer membrane) or alternatively left bare or alternatively inflated to a pre-formed shape with only size manipulatable.

Further advantages of balloon inflation devices would be operator control of x, y and z planes of the balloons thus enabling manipulation of shape as well as size in all planes to optimally and as atraumatically as possible intubate variably shaped and sized vasculature space. In some embodiments, the method of use of this invention may be similar to contemporary diagnostic and therapeutic catheters in some aspects, but with several important safety, design features and options, and therapeutic advantages associated with the present invention. For example, regarding various embodiments of the present invention, from a safety standpoint the blunt, atraumatic (non-traumatic) edge will allow traversal of all arterial vascular space much less traumatically. The blunt geometry of the present invention catheter and orifice and related will enable contact with the vascular endoluminal wall that is atraumatic. Whereas with regards to some of the drawbacks of conventional catheters, the edge of conventional catheter orifice tips may create dissection planes, lift plaque shoulders, embolize atherosclerotic debris or perforate the vessel with forward motion in arterial vascular lumens. This is due to, for example but not limited thereto, the geometry of the edge which is relatively sharp and thus capable of “digging” into the arterial vascular luminal wall with forward pressure. For example, various angulations and points of pressure will create relatively more pressure on a smaller surface area much like razor blade as opposed dichotomously to a flat surface.

However, turning to various embodiments of the present invention, a flatter more bulbous catheter orifice/tip would be much less traumatic to the arterial luminal surface. Additionally, another advantage associated with embodiments of the present invention is the capability of providing more aggressive delivery of therapeutic vascular devices for vascular luminal enlargement. For instance recent data has suggested that so called direct delivery of stents without balloon predilitation will have multiple advantages. These include less use of radiographic contrast, shorter procedure times and omission of pre-dilatation balloon injury outside of the stented arterial segment.

An advantage associated with aspects of various embodiments of the present invention device and related method is that it allows the catheter tip to be in the same plane/direction of a three-dimensional space of the ostia of the vessel and the vessel, i.e. coaxially aligned (as well as off centered to some degree if desired or required).

Direct stenting is often limited by inability to deliver the undeployed stent secondary to the frequent occurrence of the guiding catheter backing out of the vascular ostia secondary to translation of force backward from obstructing calcium, plaque and/or vascular angulation preventing forward translation of pressure. This problem of device delivery is commonly overcome by using guiding catheters with secondary and tertiary bends enabling leverage from an opposing vascular wall and so called “deep seating” the guiding catheter and/or simply using larger French guiding catheters with similar manipulations. This commonly leads to successful delivery of the device at the expense of risking vascular trauma and the associated catastrophic sequelae such as dissection leading to vessel occlusion, plaque disruption leading to thrombosis and vessel occlusion and vessel perforation all of which have a high risk of leading to death, tissue infarction, stroke, hemorrhage and circulatory collapse as well as emergency surgery. However, with regards to aspects of various embodiments of the present invention blunt tip device and/or set-back and related method the blunt tip and/or set-back prevents “deep seating” of a guide (e.g., for device delivery or other applicable procedure) while maintaining opposing vascular wall leverage obtained from pre-formed guides, i.e., prevent guide from backing out during difficult device delivery.

In contrast, another advantage associated with embodiments of the present invention is the capability of providing deflecting contrast streaming or guide wires away from the vascular luminal wall, thus further diminishing potential vascular trauma. As previously mentioned operator controlled balloon inflation devices of some embodiments of the present invention would allow manipulation of balloon tipped x, y and z planes thus enabling refined control of direction of the catheter tip/orifice and therefore direction of contrast streaming or guide wire direction. Therefore, for instance if the catheter orifice was angulated toward an ulcerated complex plaque, balloon inflation could be performed thus pushing the orifice away from the plaque. The blunt surface of the balloon tip would be relatively atraumatic to the plaque and contrast and/or guide wires would be much less likely to traumatize, dissect, perforate, etc. the vessel. With regards to various embodiments of the present invention, it is feasible that traversal of all vascular space would not even require a J tipped guide wire as the various embodiments of blunt catheter tip would effectively accomplish the same goal. This would decrease procedure cost, eliminate another step in the standard procedure sequence and thus decrease the time of the procedure. Given the extraordinary number of diagnostic and therapeutic vascular procedures performed throughout the world today a substantial amount of morbidity and mortality could be eliminated and/or diminished with this invention.

Furthermore many previously unsuccessful conventional procedures could be rendered successful due to the ability of the present invention to be more aggressive in device delivery while at the same time reducing risks of vascular trauma and its catastrophic consequences.

The subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have respiratory characteristics similar to human (e.g., cow). It should be appreciated that the subject may be any applicable patient, for example.

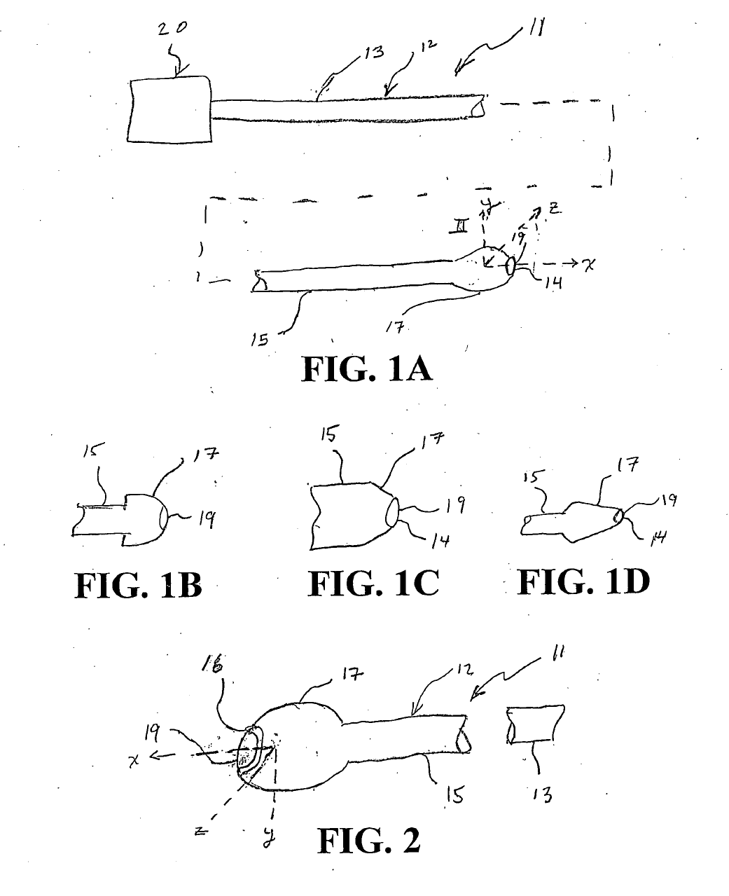

Turning to FIG. 1(A), FIG. 1(A) illustrates a schematic elevation view of an embodiment of the present invention catheter device 11 including a catheter shaft 12, interface member 20, proximal catheter portion 13, distal catheter portion 15, and a distal tip 17 having an orifice 19 defined by the lumen of the catheter therein and with an orifice edge 14 (i.e., perimeter or near perimeter or the like). The distal tip 17 is a non-traumatic (i.e., atraumatic) shape such as, but not limited thereto, any of the following: elliptical, spherical, oval, rounded, olive, bulbous, blunt, and rounded. It should be appreciated that the distal tip 17 may not necessarily be entirely elliptical or olive shaped. For example, as generally shown in FIG. 1(B), the shape of the distal tip 17 may be semi-elliptical, as well as semi-spherical, hemispherical, semi-oval, partly rounded or partly olive. The distal tip 17 provides a blunt and non-traumatic effect or interaction when the catheter shaft 12 or portion thereof and/or distal tip 17 are advanced, translated, turned or moved through the vasculature. It should be appreciated that the interface member 20 may include a number of systems and devices including, but not limited thereto, manifold, flusher, syringe, drug delivery syringe, rotor bladder, pressure manometer, etc. The interface member 20 may relate to all diagnostic intervention, pharmacological intervention and mechanical intervention systems, devices and methods.

Alternatively, as shown in FIG. 1(C), the shape of the distal tip 17 may be tapered, as well as beveled, chamfered, graduated, or multifaceted (e.g., like a diamond or the like (e.g., dodecahedron, semi-dodecahedron, icosahedron, or semi-icosahedron, etc.)). The distal tip 17 shall be tapered, beveled, chamfered, graduated or multifaceted in a manner to provide a blunt and non-traumatic effect or interaction when the catheter shaft 12 or portion thereof and/or distal tip 17 are advanced, translated, turned or moved through the vasculature.

As shown in FIG. 1(D), the shape of the distal tip 17 may be conical shaped or substantially conical shaped or the like.

The distal tip 17 may be comprised of a variety of materials including at least one of the following or combinations thereof: elastomeric, rubber, rubber-like, plastic, and polymer, as well as other materials available for catheter tips. The catheter shaft and related may be comprised of the following materials elastomeric, rubber, rubber-like, plastic, and polymer, as well as other materials available for catheters and catheter shafts.

Turning to FIG. 2, FIG. 2 is a schematic elevation view of a partial catheter device 11 including a catheter shaft 12, proximal catheter portion 13, distal catheter portion 15 and a distal tip 17 having an orifice 19 defined by the lumen of the catheter therein and with an orifice edge 14 (perimeter or near perimeter or the like). Additionally, a non-inflatable non-traumatic (i.e., atraumatic) ring or rim 16 is provided so as to, among other things, avoid or mitigate complications such as trauma, dissection and interference with the vascular walls or anatomy. This non-inflatable ring, or rim 16 may be a variety shapes such as a bumper, balloon, cylinder or tube, for example. The non-inflatable ring or rim 16 may run continuously around the circumference of the orifice 19 as illustrated, or alternatively, the non-inflatable ring or rim 16 may be semi-continuous, i.e., with individual breaks, segments or interruptions (not shown). As mentioned above, the distal tip 17 may be any one of the following such as, but not limited thereto, any of the following: elliptical, spherical, oval, rounded, olive, bulbous, and rounded. It should be appreciated that the distal tip 17 may not necessarily be entirely elliptical or olive shaped. For example, although not shown in FIG. 2, the shape of the distal tip 17 may be semi-elliptical, as well as semi-spherical, hemispherical, semi-oval, partly rounded or partly olive. Alternatively, although not shown in FIG. 2, the inflatable distal tip 17 may be tapered, beveled, chamfered, graduated, or multifaceted (e.g., like a diamond or the like).

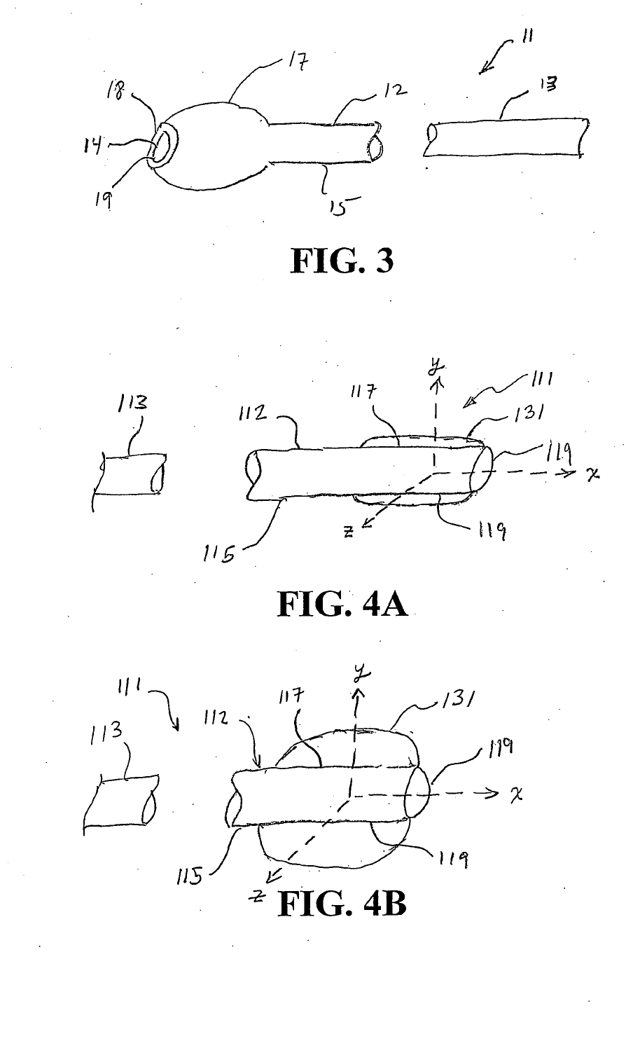

Turning to FIG. 3, FIG. 3 is a schematic elevation view of a partial catheter device 11 including a catheter shaft 12, proximal catheter portion 13, distal catheter portion 15 and a distal tip 17 having an orifice 19 defined by the lumen of the catheter therein and with an orifice edge (perimeter or near perimeter or the like). Additionally, an inflatable non-traumatic (i.e., atraumatic) ring or rim 18 is provided so as to avoid or mitigate complications such as trauma, dissection and interference with the vascular walls or anatomy. This inflatable ring or rim 18 may be a variety shapes such as a bumper, cushion, cylinder or tube, for example. The inflatable ring or rim 18 may run continuously around the circumference or perimeter of the orifice 19 as illustrated, or alternatively, the inflatable ring or rim 18 may be semi-continuous, i.e., having breaks, segments or interruptions (not shown). As mentioned above, the distal tip 17 may be any one of the following such as, but not limited thereto, any of the following: elliptical, spherical, oval, rounded, olive, bulbous, cylindrical, and rounded. It should be appreciated that the distal tip 17 may not necessarily be entirely elliptical or olive shaped. For example, although not shown in FIG. 3, the shape of the distal tip 17 may be semi-elliptical, semi-spherical, hemispherical, semi-oval, partly rounded, or partly olive. Alternatively, the distal tip 17 may be tapered, beveled, chamfered, graduated, or multifaceted (e.g., like a diamond or the like (e.g., dodecahedron, semi-dodecahedron, icosahedron, or semi-icosahedron, etc.)).

Turning to FIG. 4(A), FIG. 4(A) is a schematic elevation view of a partial catheter device 111 including a catheter shaft 112, proximal catheter portion 113, distal catheter portion 115 and a distal tip 117 having a balloon 131 (shown in a non-inflated state) or any inflatable means located proximally to or immediately at the orifice 119. Turning to FIG. 4(B), FIG. 4(B) illustrates the partial catheter device 111 of FIG. 4(A) with the balloon 131 (or any inflatable means) in an inflated state. The inflated balloon 131 (or any inflatable device) provides a blunt effect or interaction when the catheter shaft 112 or portion thereof and/or distal tip 117 are advanced, translated, turned or moved through the vasculature.

Similarly, as shown FIG. 4(C), FIG. 4(C) illustrates the partial catheter device 111 of FIG. 4(A) with the balloon 131 (or any inflatable means) in an inflated state. The inflated balloon 131 (or any inflatable device) provides a blunt effect or interaction when the catheter shaft 112 or portion thereof and/or distal tip 117 are advanced, translated, turned or moved through the vasculature. Moreover, the distal tip 117 has a set-back region or extension SB, as designated as SB, to provide a space/extension between the balloon (either inflated state or non-inflated state or both) and the very end of the distal tip 117. This set-back feature SB will provide a number of functions including, but not limited thereto, enabling the distal tip 117 to seat in the ostia of the vascular space. The set-back feature SB may enable the distal tip 117 to seat at or in the ostia at a predetermined set-back point or at an optimal/desired set back point. For instance, the balloon would therefore prevent deep seating of the distal tip 117. This set-back feature can be applied to any of the embodiments discussed throughout, such as FIGS. 1-8, indifferent of the present invention distal tips (e.g., blunt end tip, orifice (i.e., perimeter, edge, non-edge), compressible tip, inflatable rim/ring, non-inflatable rim/ring, balloon tip, etc.). For instance, the set-back region or extension SB may protrude from the blunt end contour, olive end contour, oval end contour, semi-oval end contour, etc. The set-back SB can be any desired or required dimension such as about 10 cm or more, about 5 cm or more, 1 cm or more, less than about 1 cm, or less than about 1 mm. The distance of the set-back region or extension SB may any variable length as desired or required for procedure/treatment on the subject or patient.

Turning to FIG. 4(D), FIG. 4(D) illustrates the partial catheter device 111 similarly shown FIG. 4(C) with the balloon 131 (or any inflatable means) in an inflated state. The inflated balloon 131 (or any inflatable device) provides a blunt effect or interaction when the catheter shaft 112 or portion thereof and/or distal tip 117 are advanced, translated, turned or moved through the vasculature. Moreover, the distal tip 117 has a set-back region or extension SB, as designated as SB, to provide a space/extension between the balloon (either inflated state or non-inflated state or both) and the very end of the distal tip 117. This set-back feature SB will provide a number of functions including, but not limited thereto, enabling the distal tip 117 to seat in the ostia of the vascular space. The set-back feature SB may enable the distal tip 117 to seat at or in the ostia at a predetermined set-back point or at an optimal/desired set back point. This set-back feature can be applied to any of the embodiments discussed throughout, such as FIGS. 1-8, indifferent of the present invention distal tips (e.g., blunt end tip, compressible tip, inflatable rim/ring, non-inflatable rim/ring, balloon tip, etc.).

Next, it should be appreciated that the various embodiments as discussed in throughout and referenced in FIGS. 1-8 may provide a number of possible combinations. A first, but non-limiting example, may be a catheter device as discussed throughout provided with a blunt non-traumatic distal tip without a set-back feature SB. A second, but non-limiting example, may be a catheter device as discussed throughout with a blunt non-traumatic distal tip with a set-back feature SB. A third, but non-limiting example, may be a catheter device as discussed throughout with a blunt non-traumatic distal tip with a blunt non-traumatic set-back feature SB. It should be appreciated that a blunt non-traumatic set-back element SB may be provided to incorporate or implement any and all of the non-traumatic distal tip features, advantages, designs, contours as discussed, illustrated and inferred throughout this document.

Turning to FIG. 5(A), FIG. 5(A) is a schematic elevation view of a partial catheter device 211, including a catheter shaft 212, proximal catheter portion 213, distal catheter portion 215 and a distal tip 217 having an orifice 219 wherein the distal tip 217 may have a design as discussed with the embodiments associated with anyone of FIGS. 1-4 and 6-8. In addition, the distal tip 217 (as shown in a non-compressed state in FIG. 5(A)) is made of a material that is compressible so as to be able to reduce the cross-section as desired or required. Turning to FIG. 5(B), FIG. 5(B) illustrates the partial catheter device 211 of FIG. 5(A) with the distal tip 217 in the compressed state. The properties of the compressible material may be such that it reforms to or close to its original shape, partially reforms to its original shape or does not reform to its original shape upon the removal of the given compressive forces.

Turning to FIG. 6(A), FIG. 6(A) is a schematic elevation view of a sheath 241 that has been inserted into a vasculature structure 243 such as an artery, vein, or the like. Turning to FIG. 6(B), the sheath 241 as shown in FIG. 6(A) is provided with a catheter device 211 extending there through. The compressible distal tip 217 has cross-section larger than the cross-section of the lumen of the sheath 241 or orifice 242 of the sheath 241. During use of the compressible catheter device 211 the catheter 211 is passed through the lumen of the sheath 241 in a compressed state and expands after it exits the end or orifice 242 of the sheath 241. It should be appreciated that the design of the catheter and tip may be as discussed with the embodiments associated with anyone of FIGS. 1-5 and 7-8; however the cross-section of the sheath could need to be larger to accommodate a distal tip of a catheter that is not compressible enough to fit through a narrower sheath lumen.

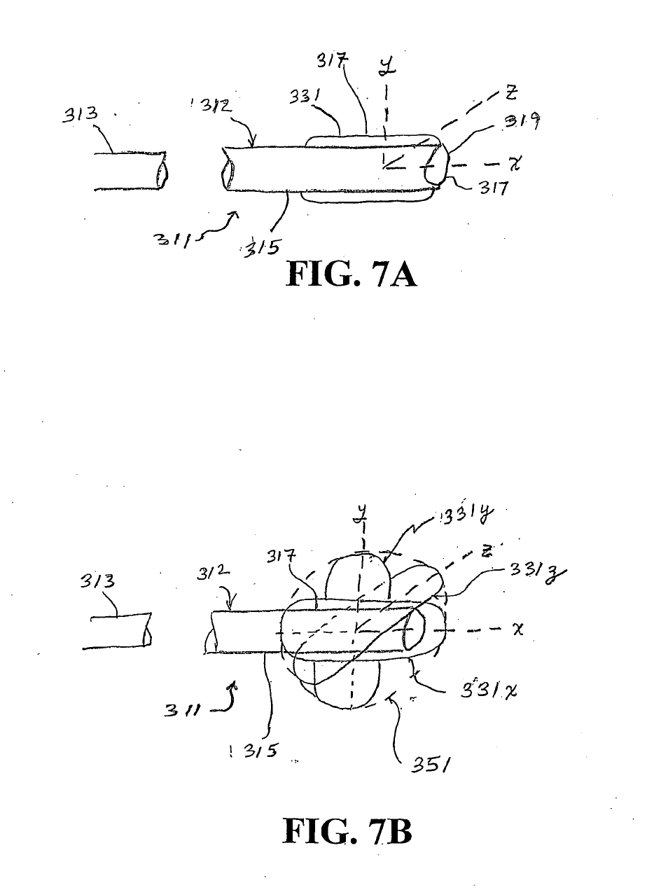

Turning to FIG. 7(A), FIG. 7(A) is a schematic elevation view of a partial catheter device 311, including a catheter shaft 312, proximal catheter portion 313, distal catheter portion 315 and a distal tip 317 having an orifice 319 wherein the distal tip 317 may have a design as discussed with the embodiments associated with anyone of FIGS. 1-6 and 8. In addition, the distal tip 317 (as shown in the non-inflated state in FIG. 6(A)) has a balloon 331 disposed or in communication with the distal tip 317. Turning to FIG. 7(B), FIG. 7(B) illustrates the partial catheter device 311 of FIG. 7(A) with the balloon 331 in an inflated state wherein the balloon 331 is adapted and designed to be inflated in a variety of shapes and contours. For example, as represented by a plurality of balloon sections 331x, 331y, 331z the overall balloon 331 may be inflated in a variety of shapes across the full geometric spectrum of potential shapes. It should be appreciated that the balloon sections 331x, 331y, 331z may be individual balloons, compartments of an overall balloon or any combination thereof. The balloons and/or compartments may be designed with the pre-formed shape concept.

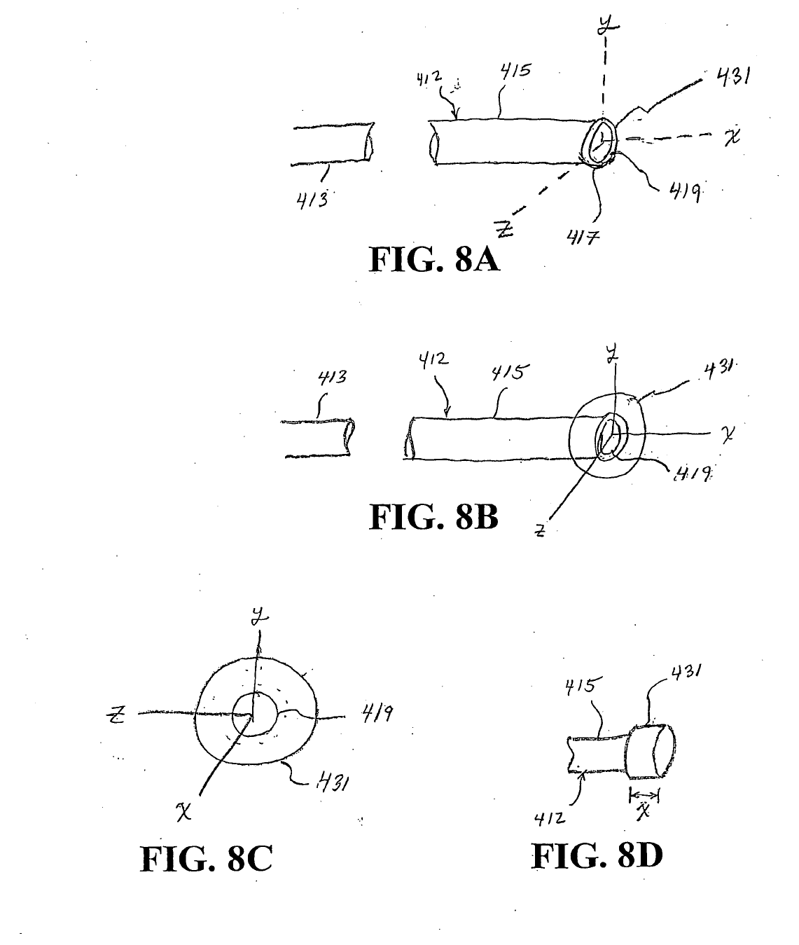

Turning to FIG. 8(A), FIG. 8(A) is a schematic elevation view of a partial catheter device 411, including a catheter shaft 412, proximal catheter portion 413, distal catheter portion 415 and a distal tip 417 having an orifice 419 wherein the distal tip 417 may have a design as discussed with the embodiments associated with anyone of FIGS. 1-7. In addition, the distal tip 317 (as shown in the non-inflated state in FIG. 8(A)) has a balloon 431 disposed or in communication with the distal tip 417. Turning to FIG. 8(1), FIG. 8(B) illustrates the partial catheter device 411 of FIG. 8(A) with the balloon 431 in an inflated state wherein the balloon 331 is adapted and designed to be inflated in a variety of shapes and contours. For example, as shown in FIG. 8(C), FIG. 8(C) is a schematic elevation end view of the catheter device 411 wherein the inflated balloon 431 is capable of expanding and retracting in the x-plane so as to make a desired or required shape, for example an oval in/out of the plane of the paper as illustrated in FIG. 8(C). The inflated balloon 431 is capable of expanding and retracting in the z-plane so as to make a desired or required shape, for example a horizontal oval as illustrated in FIG. 8(C). The inflated balloon 431 is capable of expanding and retracting in the y-plane so as to make a desired or required shape, for example a vertical oval as illustrated in FIG. 8(C).

Turning to FIG. 8(D)), FIG. 8(D) illustrates the partial catheter device 411 of FIG. 8(A) with the balloon 431 in an inflated state wherein the balloon 331 is adapted and designed to be inflated in a variety of shapes and contours. For example, as shown in FIG. 8(D), FIG. 8(D) is a schematic elevation end view of the catheter device 411 wherein the inflated balloon 431 is capable of expanding and retracting in the x-plane so as to make a desired or required shape—such a cylindrical or tubular as shown. Rather than a balloon the distal tip may comprise a compartment structure, compressible material structure, non-compressible material structure, pre-shaped balloon/compartment structure, or non-inflated structure.

In at least some of the embodiments of the present invention, during operation the distal tip of the catheter and/or other aspects of the catheter, the present invention is able to put less pressure on the shoulder of the plaque and/or vasculature walls/structures and therefore avoid or minimize the rupturing, traumatizing or injuring the plaque shoulder and/or vascular walls/structures. As a result, for example, this prevents or minimizes the release of plaque content which can lead to thrombosis or other illnesses or complications. Similarly, some embodiments of the present invention avoid or mitigate contrast from tracking between layers of artery (or vein) that causes dissection or other injuries. Accordingly, the amount of force and leverage that can be applied to a catheter and catheter tip is improved because the present invention avoids or mitigates any of the aforementioned complications, injuries or illnesses, as well as other existing complications, injuries or illnesses in the field of catheterization.

Additionally, in at least some of the embodiments of the present invention, during operation wherein a wire or other device is passed through the catheter, the present invention avoids or mitigates the occurrence of a dissection. Accordingly, the amount of force and leverage that can be applied to a catheter and catheter tip is improved because the present invention avoids or mitigates any of the aforementioned complications, injuries or illnesses, as well as other existing complications, injuries or illnesses in the field of catheterization.

The invention may be embodied in other specific forms without departing from the spirit or essential characteristics thereof. The foregoing embodiments are therefore to be considered in all respects illustrative rather than limiting of the invention described herein.

One skilled in the art can appreciate that many other embodiments of catheter device, and other details of construction constitute non-inventive variations of the novel and insightful conceptual means, system and technique which underlie the present invention.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority-hereto, then any such conflicting information in such incorporated by reference material is specifically not incorporated by reference herein.

We claim:

1. A catheter device for diagnostic vascular treatment and/or therapeutic vascular treatment of a subject's vasculature, said device comprising:
a catheter shaft having a proximal portion and a distal portion; and
a distal tip disposed on said distal portion, said distal tip having a blunt shape adapted to avoid or mitigate trauma with an ostium of the vasculature.

2. The catheter device of claim 1, wherein said distal tip is adapted to be compressible to fit through a sheath or other conduit.

3. The catheter device of claim 1, wherein said distal tip is inflatable.

4. The catheter device of claim 1, wherein said distal tip has at least one the following shapes: olive, bulbous, rounded, spherical, hemispherical, conical, oval, tapered, beveled, chamfered, graduated and/or multi-faceted, or any combination thereof.

5. The device of claim 4, wherein said distal tip comprises a set-back extension located on the distal end of said distal tip.

6. The device of claim 5, wherein said set-back extension having a blunt shape adapted to avoid or mitigate trauma with an ostium of the vasculature.

7. The catheter device of claim 6, wherein said set-back extension has at least one the following shapes: olive, bulbous, rounded, spherical, hemispherical, conical, oval, tapered, beveled, chamfered, graduated and/or multi-faceted, or any combination thereof.

8. The device of claim 5, wherein said set-back extension has at least one the following shapes: semi-elliptical, semi-spherical, hemispherical, semi-oval, partly rounded or partly olive, or any combination thereof.

9. The device of claim 5, wherein said set-back extension is adapted to be manipulated along the entire geometric spectrum of potential shapes to create non-traumatic tip.

10. The catheter device of claim 5, further comprising:
a set-back balloon disposed on said set-back extension that is inflatable.

11. The catheter device of claim 10, wherein said set-back balloon has a pre-formed shape for inflation.

12. The device of claim 1, wherein said distal tip comprises a set-back off extension located on the distal end of said distal tip.

13. The device of claim 12, wherein said set-back extension having a blunt shape adapted to avoid or mitigate trauma with an ostium of the vasculature.

14. The catheter device of claim 13, wherein said set-back extension has at least one the following shapes: olive, bulbous, rounded, spherical, hemispherical, conical, oval, tapered, beveled, chamfered, graduated and/or multi-faceted, or any combination thereof.

15. The device of claim 12, wherein said set-back extension has at least one the following shapes: semi-elliptical, semi-spherical, hemispherical, semi-oval, partly rounded or partly olive, or any combination thereof.

16. The device of claim 12, wherein said set-back extension is adapted to be manipulated along the entire geometric spectrum of potential shapes to create non-traumatic tip.

17. The catheter device of claim 12, further comprising:
a set-back balloon disposed on said set-back extension that is inflatable.

18. The catheter device of claim 17, wherein said set-back balloon has a pre-formed shape for inflation.

19. The device of claim 1, wherein said distal tip has at least one the following shapes: semi-elliptical, semi-spherical, hemispherical, semi-oval, partly rounded or partly olive, or any combination thereof.

20. The device of claim 1, wherein said distal tip is adapted to be manipulated along the entire geometric spectrum of potential shapes to create non-traumatic tip.

21. The catheter device of claim 1, further comprising:
a balloon disposed on said distal tip that is inflatable.

22. The catheter device of claim 21, wherein said balloon has at least one the following shapes when at least partially inflated: olive, bulbous, rounded, spherical, hemispherical, conical, oval, tapered, beveled, chamfered, graduated and/or multi-faceted, or any combination thereof.

23. The catheter device of claim 21, wherein said balloon has at least one the following shapes when at least partially inflated: cylindrical, tubular or ring-like.

24. The catheter device of claim 21, wherein said balloon has a pre-formed shape for inflation.

25. The device of claim 21, wherein said distal tip comprises a set-back extension located on the distal end of said distal tip located distally from said balloon.

26. The device of claim 25, wherein said set-back extension having a blunt shape adapted to avoid or mitigate trauma with an ostium of the vasculature.

27. The catheter device of claim 26, wherein said distal tip has at least one the following shapes: olive, bulbous, rounded, spherical, hemispherical, conical, oval, tapered, beveled, chamfered, graduated and/or multi-faceted, or any combination thereof.

28. The device of claim 25, wherein when said balloon is in an inflated state said distal tip forms a non-traumatic shape.

29. The catheter device of claim 1, further comprising:
a plurality of balloons disposed on said distal tip is inflatable.

30. The device of claim 29, wherein said distal tip comprises a set-back extension located on the distal end of said distal tip located distally from at least one of said balloons.

31. The device of claim 30, wherein said set-back extension having a blunt shape adapted to avoid or mitigate trauma with an ostium of the vasculature.

32. The catheter device of claim 31, wherein said set-back extension has at least one the following shapes: olive, bulbous, rounded, spherical, hemispherical, conical, oval, tapered, beveled, chamfered, graduated and/or multi-faceted, or any combination thereof.

33. The device of claim 29, wherein when at least some of said balloons are in an inflated state said distal tip forms a non-traumatic shape.

34. The device of any one of claims 5, 12, 25, or 30, wherein said set-back extension is greater than 10 cm.

35. The device of any one of claims 5, 12, 25, or 30, wherein said set-back extension is less than 10 cm.

36. The device of any one of claims 5, 12, 25, or 30, wherein said set-back extension is about 2 cm.

37. The device of any one of claims 5, 12, 25, or 30, wherein said set-back extension is about 1 cm.

38. The device of any one of claims 5, 12, 25, or 30, wherein said set-back extension is between about 1 cm and about 5 mm.

39. The device of any one of claims 5, 12, 25, or 30, wherein said set-back extension is less than about 5 mm.

40. The device of any one of claims 5, 12, 25, or 30, wherein said set-back extension is about 1 mm.

41. The device of claim 1, wherein the vascular diagnostic treatment comprises an invasive procedure in which the catheter device and related are passed into a peripheral vein or artery, through the blood vessels, and into the heart or other vasculature.

42. The device of claim 1, wherein the vascular diagnostic treatment comprises at least one of: coronary and peripheral vasculature angiography or coronary arteriography and angiography.

43. The device of claim 1, wherein the vascular therapeutic treatment comprises therapeutic cardiac catheterization including at least one of the following: percutaneous transluminal angioplasty (PTA) (alternatively, percutaneous transluminal coronary angioplasty (PTCA)), percutaneous coronary intervention (PCI), and percutaneous transluminal interventions (PTI).

44. The device of claim 1, wherein the vascular therapeutic treatment provides improved leverage for delivery of therapeutic interventional hardware.

45. The device of claim 44, wherein the hardware comprises at least one of balloons, stents, atherectomy devices, lasers, or thrombectomy devices.

46. The device of claim 1, wherein said catheter shaft comprises a lumen defining an orifice disposed on said distal tip.

47. The device of claim 46, wherein said orifice comprises a perimeter that is substantially rounded creating a smooth, non-edged orifice interface with the subject's vasculature.

48. The device of claim 1, wherein the blunt shape of said distal tip prevents deep seating of a guide that is being used in said catheter device during treatment while maintaining opposing vascular wall leverage obtained from pre-formed guides.

49. A method of performing diagnostic vascular treatment and/or therapeutic vascular treatment on a subject's vasculature using a catheter device, wherein said catheter device comprises:
a catheter shaft having a proximal portion and a distal portion; and
a distal tip disposed on said distal portion, said distal tip having a blunt shape adapted to avoid or mitigate trauma with an ostium of the vasculature.

50. The method of performing diagnostic vascular treatment and/or therapeutic vascular treatment using a catheter device according to any one of claims 1-33 or 41-48.

51. The method of performing diagnostic vascular treatment and/or therapeutic vascular treatment using a catheter device according to claim 34.

52. The method of performing diagnostic vascular treatment and/or therapeutic vascular treatment using a catheter device according to claim 35.

53. The method of performing diagnostic vascular treatment and/or therapeutic vascular treatment using a catheter device according to claim 36.

54. The method of performing diagnostic vascular treatment and/or therapeutic vascular treatment using a catheter device according to claim 37.

55. The method of performing diagnostic vascular treatment and/or therapeutic vascular treatment using a catheter device according to claim 38.

56. The method of performing diagnostic vascular treatment and/or therapeutic vascular treatment using a catheter device according to claim 39.

57. The method of performing diagnostic vascular treatment and/or therapeutic vascular treatment using a catheter device according to claim 40.
